(19) United States
(12) Patent Application Publication
(10) Pub. No.: US 2026/0083983 A1
(43) Pub. Date: Mar. 26, 2026

(54) CONCURRENT OPTIMIZATION OF PARTICLE EMITTER SYSTEM FOR MULTI-PHASED TREATMENT

(71) Applicant: Siemens Healthineers International AG, Steinhausen (CH)

(72) Inventors: Roni Hytonen, Espoo (FI); Perttu Niemelä, Espoo (FI); Lauri Halko, Helsinki (FI)

(21) Appl. No.: 18/898,510

(22) Filed: Sep. 26, 2024

(51) Int. Cl. A61N 5/10; G16H 20/40

(52) U.S. Cl. CPC A61N 5/1031; G16H 20/40; A61N 2005/1087

(57) ABSTRACT

Aspects of this technical solution can determine a first dose of radiation concurrently with a second dose of radiation, the first dose associated with a first phase of operation of a particle emitter device, and the second dose associated with a second phase of operation of a particle emitter device, generate a first phase metric concurrently with a second phase metric, the first phase metric for a first objective on the first dose during the first phase, and the second phase metric for a second objective on the second dose during the second phase, determine, based on the first dose and the second dose, a combined dose over the first phase and the second phase, modify, at least one of the first dose or the second dose to reduce the combined metric, and cause the particle emitter device to emit the first dose and the second dose.

### TECHNICAL FIELD

The present implementations relate generally to radiation therapy, including but not limited to concurrent optimization of particle emitter systems for multi-phased treatment.

### BACKGROUND

Radiation therapy is becoming increasingly desired in the treatment of medical conditions and illnesses. Accurate dosage of radiation is becoming increasingly complex as the number and types of techniques and radiation capabilities increase. However, conventional systems cannot effectively or accurately determine accurate dosage. For example, conventional base-dose planning, where the dose from the first phase is incorporated into the optimization process of the second phase, cannot optimize across multiple phases.

### SUMMARY

The methods and systems discussed herein provide technical solutions directed at least to providing concurrent or simultaneous optimization of a plurality of treatment plans, each capable of including a plurality of doses. In an aspect, this technical solution allows concurrent optimization of different modalities of radiation therapy, including but not limited to any combination of photos or protons. Thus, a technical solution for concurrent optimization of particle emitter systems for multi-phased treatment is provided. This technical solution can provide a technical improvement to significantly streamline and simplify the planning process to achieve higher granularity of dose delivery beyond the capability of manual processes to achieve.

The methods and systems discussed herein provide simultaneous optimization of multi-phased treatment plans in radiation therapy. The methods and systems discussed herein integrate control and feedback mechanisms to streamline the planning process, enabling efficient and effective treatment delivery. The systems and methods discussed herein include a user interface that allows users to define optimization objectives for each phase of treatment and the combined plan sum. The multi-phase optimization algorithm discussed herein may calculate doses for each phase, evaluating how well they meet the set objectives, and iteratively adjust the treatment fields to minimize the total cost, ensuring optimal dose distribution. This approach addresses the inefficiencies and suboptimal outcomes of traditional separate planning methods by enabling real-time adjustments and reducing the need for iterative re-planning, ultimately improving the efficiency and effectiveness of radiation treatment planning.

In some aspects, the techniques described herein relate to a system, including: one or more processors, coupled with memory, to: determine a first dose of radiation concurrently with a second dose of radiation, the first dose of radiation associated with a first phase of operation of a particle emitter device, and the second dose of radiation associated with a second phase of operation of a particle emitter device, the second phase subsequent to the first phase; generate a first phase metric concurrently with a second phase metric, the first phase metric corresponding to a first objective on the first dose of radiation during the first phase, and the second phase metric corresponding to a second objective on the second dose of radiation during the second phase; determine, based on the first dose of radiation and the second dose of radiation, a combined dose of radiation over the first phase and the second phase; generate, based on the first dose of radiation and the second dose of radiation, a combined metric corresponding to a total cost of radiation over the first phase and the second phase; modify, based on the combined metric, at least one of the first dose of radiation or the second dose of radiation to reduce the combined metric; and cause the particle emitter device to emit the first dose of radiation and the second dose of radiation according to the modification.

In some aspects, the techniques described herein relate to a system, the processors to: generate, based on the combined dose of radiation and a third objective on the combined dose of radiation, a second combined metric corresponding to a cost of radiation independent of the first phase and the second phase.

In some aspects, the techniques described herein relate to a system, the processors to: generate, based on the second combined metric, the combined metric corresponding to the total cost of radiation.

In some aspects, the techniques described herein relate to a system, the processors to: modify at least one of the first dose of radiation or the second dose of radiation to reduce the combined metric over one or more subsequent iterations.

In some aspects, the techniques described herein relate to a system, the processors to: cause a user interface to present one or more first indications of the first dose of radiation over the one or more subsequent iterations; and cause the user interface to present one or more second indications of the second dose of radiation over the one or more subsequent iterations overlaid with the one or more first indications.

In some aspects, the techniques described herein relate to a system, the processors to: cause the user interface to present one or more third indications of the combined dose of radiation over the one or more subsequent iterations overlaid with the one or more first indications and the one or more second indications.

In some aspects, the techniques described herein relate to a system, wherein the particle emitter device is configured to provide, during the first phase, the first dose of radiation via a first plurality of fields.

In some aspects, the techniques described herein relate to a system, wherein the particle emitter device is configured to provide, during the second phase, the second dose of radiation via a second plurality of fields.

In some aspects, the techniques described herein relate to a method, including: determining a first dose of radiation concurrently with a second dose of radiation, the first dose of radiation associated with a first phase of operation of a particle emitter device, and the second dose of radiation associated with a second phase of operation of a particle emitter device, the second phase subsequent to the first phase; generating a first phase metric concurrently with a second phase metric, the first phase metric corresponding to a first objective on the first dose of radiation during the first phase, and the second phase metric corresponding to a second objective on the second dose of radiation during the second phase; determining, based on the first dose of radiation and the second dose of radiation, a combined dose of radiation over the first phase and the second phase; generating, based on the first dose of radiation and the second dose of radiation, a combined metric corresponding to a total cost of radiation over the first phase and the second phase; modifying, based on the combined metric, at least one of the first dose of radiation or the second dose of radiation to reduce the combined metric; and causing the particle emitter device to emit the first dose of radiation and the second dose of radiation according to the modification.

In some aspects, the techniques described herein relate to a method, further including: generating, based on the combined dose of radiation and a third objective on the combined dose of radiation, a second combined metric corresponding to a cost of radiation independent of the first phase and the second phase.

In some aspects, the techniques described herein relate to a method, further including: generating, based on the second combined metric, the combined metric corresponding to the total cost of radiation.

In some aspects, the techniques described herein relate to a method, further including: modifying at least one of the first dose of radiation or the second dose of radiation to reduce the combined metric over one or more subsequent iterations.

In some aspects, the techniques described herein relate to a method, further including: causing a user interface to present one or more first indications of the first dose of radiation over the one or more subsequent iterations; and causing the user interface to present one or more second indications of the second dose of radiation over the one or more subsequent iterations overlaid with the one or more first indications.

In some aspects, the techniques described herein relate to a method, further including: causing the user interface to present one or more third indications of the combined dose of radiation over the one or more subsequent iterations overlaid with the one or more first indications and the one or more second indications.

In some aspects, the techniques described herein relate to a method, wherein the particle emitter device is configured to provide, during the first phase, the first dose of radiation via a first plurality of fields.

In some aspects, the techniques described herein relate to a method, wherein the particle emitter device is configured to provide, during the second phase, the second dose of radiation via a second plurality of fields.

In some aspects, the techniques described herein relate to a non-transitory computer readable medium including one or more instructions stored thereon and executable by a processor to: determine, by the processor, a first dose of radiation concurrently with a second dose of radiation, the first dose of radiation associated with a first phase of operation of a particle emitter device, and the second dose of radiation associated with a second phase of operation of a particle emitter device, the second phase subsequent to the first phase; generate, by the processor, a first phase metric concurrently with a second phase metric, the first phase metric corresponding to a first objective on the first dose of radiation during the first phase, and the second phase metric corresponding to a second objective on the second dose of radiation during the second phase; determine, by the processor and based on the first dose of radiation and the second dose of radiation, a combined dose of radiation over the first phase and the second phase; generate, by the processor and based on the first dose of radiation and the second dose of radiation, a combined metric corresponding to a total cost of radiation over the first phase and the second phase; modify, by the processor and based on the combined metric, at least one of the first dose of radiation or the second dose of radiation to reduce the combined metric; and cause, by the processor, the particle emitter device to emit the first dose of radiation and the second dose of radiation according to the modification.

In some aspects, the techniques described herein relate to a non-transitory computer readable medium, further including one or more instructions executable by the processor to: generate, by the processor and based on the combined dose of radiation and a third objective on the combined dose of radiation, a second combined metric corresponding to a cost of radiation independent of the first phase and the second phase.

In some aspects, the techniques described herein relate to a non-transitory computer readable medium, further including one or more instructions executable by the processor to: generate, by the processor and based on the second combined metric, the combined metric corresponding to the total cost of radiation.

In some aspects, the techniques described herein relate to a non-transitory computer readable medium, further including one or more instructions executable by the processor to: modify, by the processor, at least one of the first dose of radiation or the second dose of radiation to reduce the combined metric over one or more subsequent iterations.

### BRIEF DESCRIPTION OF THE FIGURES

These and other aspects and features of the present implementations are depicted by way of example in the figures discussed herein. Present implementations can be directed to, but are not limited to, examples depicted in the figures discussed herein. Thus, this disclosure is not limited to any figure or portion thereof depicted or referenced herein, or any aspect described herein with respect to any figures depicted or referenced herein.

FIG. 1A depicts an example user interface presentation of first phase treatment, according to some embodiments.

FIG. 1B depicts an example user interface presentation of second phase treatment, according to some embodiments.

FIG. 2 depicts an example system, according to some embodiments.

FIG. 3A depicts an example user interface presentation of first phase optimization, according to some embodiments.

FIG. 3B depicts an example user interface presentation of second phase optimization, according to some embodiments.

FIG. 3C depicts an example user interface presentation of a summation of the phases optimization, according to some embodiments.

FIG. 4 depicts an example computer architecture for dose optimization, according to some embodiments.

FIG. 5 depicts an example method of concurrent optimization of particle emitter system for multi-phased treatment, according to some embodiments.

FIG. 6 depicts an example method of concurrent optimization of particle emitter system for multi-phased treatment, according to some embodiments.

### DETAILED DESCRIPTION

Aspects of this technical solution are described herein with reference to the figures, which are illustrative examples of this technical solution. The figures and examples below are not meant to limit the scope of this technical solution to the present implementations or a single implementation, and other implementations in accordance with present implementations are possible, for example, by way of interchange of some or all of the described or illustrated elements. Where certain elements of the present implementations can be partially or fully implemented using known components, only those portions of such known components that are necessary for an understanding of the present implementations are described, and detailed descriptions of other portions of such known components are omitted not to obscure the present implementations. Terms in the specification and claims are to be ascribed no uncommon or special meaning unless explicitly set forth herein. Further, this technical solution and the present implementations encompass present and future known equivalents to the known components referred to herein by way of description, illustration, or example.

In radiation treatment planning, it is advantageous to simultaneously create multiple treatment plans for a patient who is treated in multiple phases. For example, a patient may first be treated with a course of radiation to a larger ‘elective’ volume (or a first phase), followed by another course to a smaller ‘boost’ volume within the elective volume (or a second phase, such as 326A). The phases can consist either of the same treatment modality or a mixture of different modalities (e.g., proton/photon/Flash). A total treatment plan can include a first treatment plan for a first phase and a second treatment plan for a second phase plans, and can optimize (e.g., minimize) total dose to organs at risk (OAR) for the sum of the two plans. Thus, this technical solution is directed at least to the concurrent optimization of multi-phased treatment plans, which allows a user to easily define optimization objectives for each phase and for a planned sum, as well as to evaluate a dose for each phase during a single optimization session. This solution can include a feedback-control system and a multi-phase optimization architecture. Though this disclosure illustrates two intensity-modulated proton planning phases, it is not limited thereto. For example, this technical solution is applicable to any number of phases with any number of fields or modalities (e.g., IMPT, Flash, or photon).

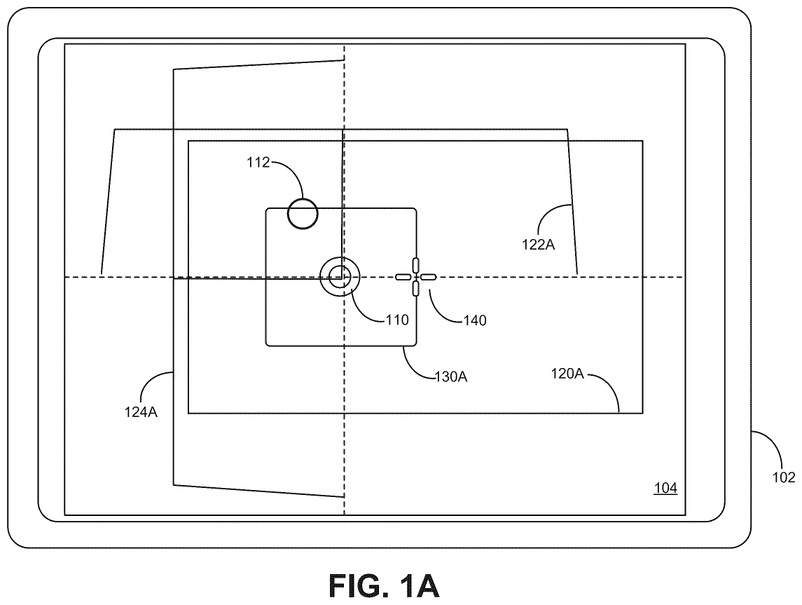

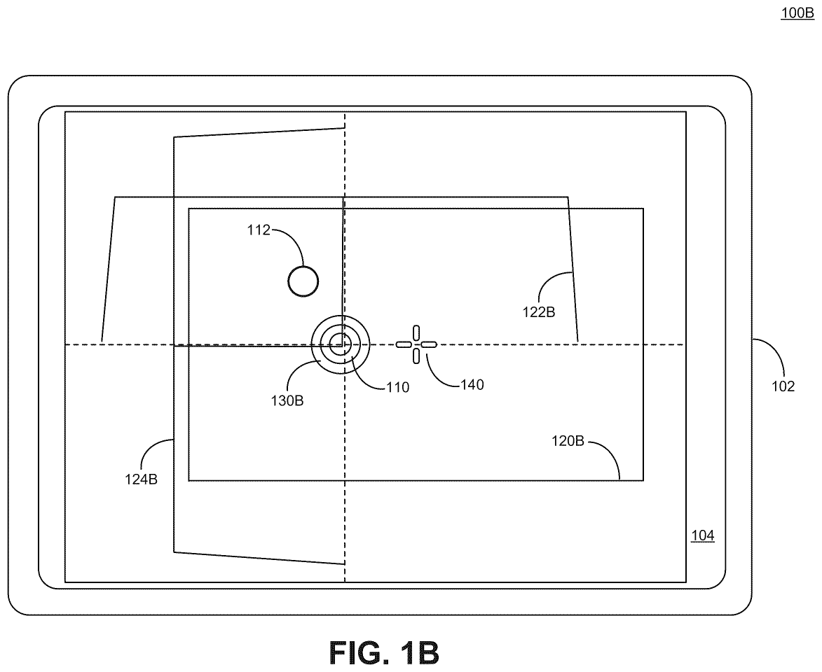

FIGS. 1A-B are directed at least to user interfaces to present dose optimization and control feedback for dose optimization. In some embodiments, user interfaces, as discussed herein, can concurrently or simultaneously provide a view of and controls over different phases, as well as the sum of phases in an individual, self-contained optimization dialogue. In an aspect, a user interface can include separate views for each phase and the sum of the phases. For example, optimization objectives defined in any of the views can be applied to that phase, and each view can show the feedback (DVH, dose) for that phase. This way, there is no need to switch between phase plans or to calculate separate plan sums to finish the optimization process. For example, the phase plans are actual deliverable plans. In contrast, the plan sum is a transient plan that only exists within the optimization context to facilitate setting the sum objectives and visualizing the sum doses. Outside the optimization dialogue, the sum plan can be recreated as a plan sum of the different phases. For example, a first phase targets PTV1 with two fields, and a second phase targets PTV2 with two distinct fields A and B.

FIGS. 1A-B illustrate an example corresponding to one patient for whom a two-phased proton-based treatment is prescribed. In the first phase, 5 Gy of radiation is directed to volume PTV1. In the second phase, an additional 2 Gy is directed to volume PTV2, which is enclosed within PTV1. In addition, the total mean dose received by the OAR from both phases is minimized. As the number of phases and fields increases for increasingly complex treatment plans, there can be significant iterating to ensure that both phases and their sum yield desirable dose distributions. Thus, this technical solution can provide a technical improvement to generate a treatment plan for radiation therapy via a particle emitter system beyond the capability of manual processes (or conventional computer-implemented processes) to achieve. The technical solution can also configure the particle emitter system according to the generated treatment plan.

FIG. 1A depicts an example user interface presentation of first phase treatment, according to an embodiment. As illustrated by way of example in FIG. 1A, a user interface presentation of first phase treatment 100A can include at least a display 102. The display 102 can display at least one or more presentations, as discussed herein, and can include an electronic display.

An electronic display can include, for example, a liquid crystal display (LCD), a light-emitting diode (LED) display, an organic light-emitting diode (OLED) display, or the like. The display device can receive, for example, capacitive or resistive touch input. The display 102 can include a user interface 104. The user interface 104 can be at least partially presented at or by the display 102 to receive input from a user or to provide output to a user. For example, user interface 104 can correspond to a display device that provides visual output to a user and one or more user input devices that receive input from a user. For example, the input devices can include a keyboard, mouse, or touch-sensitive panel of the display device, but they are not limited thereto. The user interface 104 can include a target indicator 110, a secondary target indicator 112, a treatment frame 120A, a target frame 130A, and a cursor 140.

The target indicator 110 can correspond to a focal point for radiation therapy during the first phase. For example, the target indicator 110 can identify a portion of the surface of the body of a patient on which a radiation emission is to be centered. The secondary target indicator 112 can correspond to a second focal point for radiation therapy during the first phase that is distinct from the focal point corresponding to the target indicator 110. For example, the secondary target indicator 112 can identify a second portion of the surface of the body of a patient on which a radiation emission is to be centered. For example, the target indicator 110 and the secondary target indicator 112 can be part of a sequence of focal points traversing the surface of the body of the patient.

The treatment frame 120A can identify a region of the surface of the body of the patient corresponding to the first phase of treatment, including one or more of the target indicator 110 and the secondary target indicator 112. The treatment frame 120A can include a first field 122A and a second field 124A. The first field 122A can correspond to a first radiation emission focused according to the target indicator 110. For example, the first radiation emission can have first properties (e.g., direction, modality, power, period of activation, duty cycle of activation, or any combination thereof). The second field, 124A, can correspond to a second radiation emission focused according to the target indicator 110. For example, the second radiation emission can have second properties, as discussed herein, but they are not limited thereto. For example, one or more of the second properties can differ from the first properties. The target frame 130A can identify a region of the surface of the body of the patient to which the first radiation emission and the second radiation emission are applied. For example, the target frame 130A can correspond to an elective volume as discussed herein but is not limited thereto.

FIG. 1B depicts an example user interface presentation of the second phase treatment, according to this disclosure. As illustrated by way of example in FIG. 1B, a user interface presentation of second phase treatment 100B can include at least a treatment frame 120B and a target frame 130B. The treatment frame 120B can identify a region of the surface of the patient's body corresponding to the second phase of treatment, including one or more of the target indicator 110 and the secondary target indicator 112. The treatment frame 120B can include a first field 122B and a second field 124B. The first field, 122B, can correspond to a third radiation emission focused according to the target indicator 110. For example, the third radiation emission can have third properties, as discussed herein, but it is not limited thereto. For example, one or more of the third properties can differ from the first properties or the second properties.

The second field, 124B, can correspond to a fourth radiation emission focused according to the target indicator 110. For example, the fourth radiation emission can have fourth properties as discussed herein but is not limited thereto. For example, one or more of the fourth properties can differ from the first properties, the second properties, or the third properties. The target frame 130B can identify a region of the surface of the body of the patient to which the third radiation emission and the fourth radiation emission are applied. For example, the target frame 130B can correspond to a boost volume as discussed herein but is not limited thereto.

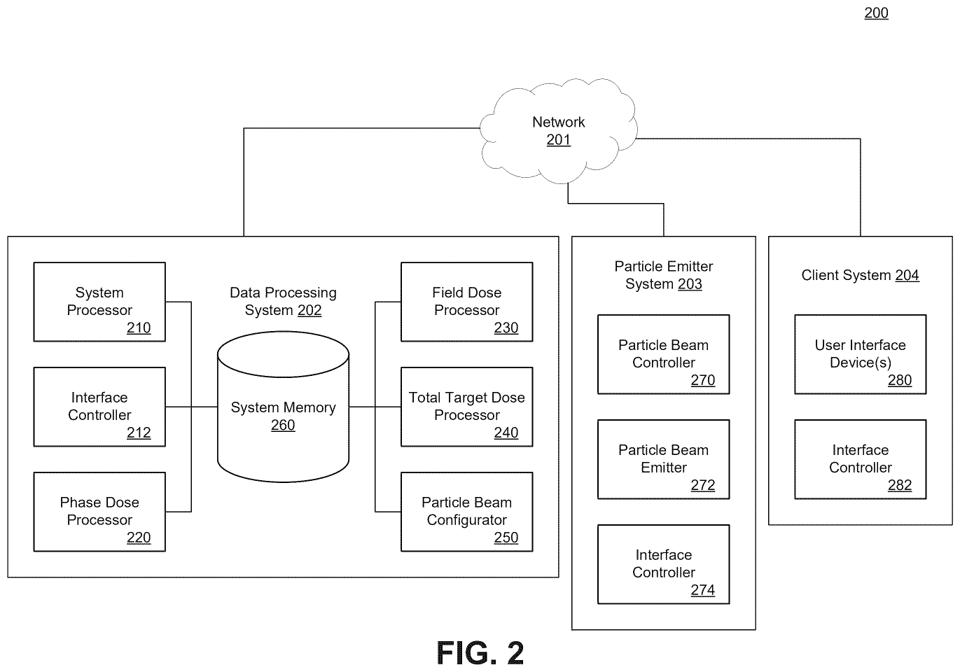

FIG. 2 depicts an example system, according to this disclosure. As illustrated by way of example in FIG. 2, a system 200 can include at least a network 201, a data processing system 202, a particle emitter system 203, and a client system 204.

The network 101 can include any type or form of network. The geographical scope of the network 101 can vary widely and the network 101 can include a body area network (BAN), a personal area network (PAN), a local-area network (LAN), e.g., Intranet, a metropolitan area network (MAN), a wide area network (WAN), or the Internet. The topology of the network 101 can be of any form and can include, e.g., any of the following: point-to-point, bus, star, ring, mesh, or tree. The network 101 can include an overlay network which is virtual and sits on top of one or more layers of other networks 101. The network 101 can be of any such network topology as known to those ordinarily skilled in the art capable of supporting the operations described herein. The network 101 can utilize different techniques and layers or stacks of protocols, including, e.g., the Ethernet protocol, the Internet protocol suite (TCP/IP), the ATM (Asynchronous Transfer Mode) technique, the SONET (Synchronous Optical Networking) protocol, or the SD (Synchronous Digital Hierarchy) protocol. The ′TCP/IP Internet protocol suite can include the application layer, transport layer, Internet layer (including, e.g., IPv6), or the link layer. The network 101 can include a type of a broadcast network, a telecommunications network, a data communication network, or a computer network.

The data processing system 202 can include a physical computer system operatively coupled or couplable with one or more components of the system 100, either directly or indirectly through an intermediate computing device or system. The data processing system 202 can include a virtual computing system, an operating system, and a communication bus to affect communication and processing. The data processing system 202 can include a system processor 210, an interface controller 212, a phase dose processor 220, a field dose processor 230, a total target dose processor 240, a particle beam configurator 250, and a system memory 260.

The system processor 210 can execute one or more instructions associated with the system 100. The system processor 210 can include an electronic processor, an integrated circuit, or the like including one or more of digital logic, analog logic, digital sensors, analog sensors, communication buses, volatile memory, nonvolatile memory, and the like. The system processor 210 can include but is not limited to, at least one microcontroller unit (MCU), a microprocessor unit (MPU), a central processing unit (CPU), a graphics processing unit (GPU), a physics processing unit (PPU), an embedded controller (EC), or the like. The system processor 210 can include a memory operable to store or storing one or more instructions for operating components of the system processor 210 and operating components operably coupled to the system processor 210. For example, one or more instructions can include one or more firmware, software, hardware, operating systems, or embedded operating systems. The system processor 210 or the system 100 generally can include one or more communication bus controllers to effect communication between the system processor 210 and the other elements of the system 100.

The interface controller 212 can link the data processing system 202 with one or more of the network 201 and the client system 204 by one or more communication interfaces. A communication interface can include, for example, an application programming interface (“API”) compatible with a particular component of the data processing system 202 or the client system 204. The communication interface can provide a particular communication protocol compatible with a particular component of the data processing system 202 and a particular component of the client system 204. The interface controller 212 can be compatible with particular content objects and can be compatible with particular content delivery systems corresponding to particular content objects, structures of data, types of data, or any combination thereof. For example, the interface controller 212 can be compatible with the transmission of text data or binary data structured according to one or more metrics or data of the client system 204.

The first phase dose processor 220 can generate treatment attributes associated with a radiotherapy treatment of a patient (e.g., dose distributions) based on patient data and other data inputted by the end-user. The second phase, dose processor 230, can generate treatment attributes associated with a radiotherapy treatment of a patient (e.g., dose distributions) based on patient data and other data inputted by the end-user. The total target dose processor 240 can combine the dosages and other treatment attributes calculated by the first phase dose processor 220 and second dose processor 230. The particle beam configurator 250 can change one or more configurations of a radiation therapy machine in accordance with the treatment plan generated using the methods and systems discussed herein.

The system memory 260 can store data associated with the system 100. The system memory 260 can include one or more hardware memory devices to store binary data, digital data, or the like. The system memory 260 can include one or more electrical components, electronic components, programmable electronic components, reprogrammable electronic components, integrated circuits, semiconductor devices, flip flops, arithmetic units, or the like. The system memory 260 can include at least one non-volatile memory device, a solid-state memory device, a flash memory device, or a NAND memory device. The system memory 260 can include one or more addressable memory regions disposed on one or more physical memory arrays.

A physical memory array can include a NAND gate array disposed on, for example, at least one of a particular semiconductor device, integrated circuit device, and printed circuit board device. In an aspect, the system memory 260 can correspond to a non-transitory computer-readable medium. For example, the non-transitory computer-readable medium can store instructions corresponding to one or more of the components of system 100, as discussed herein, but it is not limited thereto. In an aspect, the non-transitory computer-readable medium can include one or more instructions executable by the system processor. The processor can generate, based on the combined dose of radiation and a third objective on the combined dose of radiation, a second combined metric corresponding to a cost of radiation independent of the first phase and the second phase. In an aspect, the processor can generate, based on the second combined metric, the combined metric corresponding to the total cost of radiation. In an aspect, the processor can modify at least one of the first doses of radiation or the second dose of radiation to reduce the combined metric over one or more subsequent iterations.

The particle emitter system 203 is or includes a radiation output system operable to apply directed energy to a target. A target may include a biological organism. As one example, a biological organism can be a person, animal, or the like. As another example, a person can be a patient undergoing radiation therapy treatment in accordance with directed energy applied from the particle emitter system 203 to at least a portion of a body, body part, or the like of the patient. The particle emitter system 203 may apply at least one proton beam having at least one distribution pattern corresponding to one or more operating states of one or more components thereof or associated therewith. The particle emitter system 203 may include, correspond to, or be associated with, or the like, a patient treatment room of a medical facility at the clinical location 120. The patient treatment room of the medical facility can correspond to a room, a radiology facility, or the like, of a hospital, medical facility, clinic, or the like. The particle emitter system 203 can include a particle beam controller 270, a particle beam emitter 272, and an interface controller 274.

The particle emitter system 203 may be or may include one or more moveable, articulable, or like components thereof or associated therewith. The particle emitter system 203 can include at least one nozzle, including a beam output component. As one example, the nozzle can be a scanning nozzle operable to have a first output characteristic corresponding to a first output aperture for directing a proton beam. As another example, the first output aperture can correspond to a proton beam shape having a size, energy, current, and the like compatible with the application of a proton beam to the living tissue of a biological organism, patient, and the like. As another example, the nozzle can be an eye nozzle operable to have a second output characteristic corresponding to a second output aperture for directing a proton beam. As another example, the second output aperture can correspond to a proton beam shape having a size, energy, current, and the like compatible with the application of a proton beam to living ocular tissue of a biological organism, patient, and the like. Each nozzle can correspond to or generate output according to a field, as discussed herein.

The particle beam controller 270 may be a device or a software module used in radiation therapy to precisely manage and direct particle beams, such as proton or heavy ion beams, toward target tissues within a patient's body. The particle beam emitter 272 can include a radiation-generating system operable to generate directed energy. The particle beam emitter 272 can be or can include a cyclotron operable to generate one or more focused energy beams, including one or more proton beams or the like. The particle beam emitter 272 can generate at least one proton beam having at least one distribution pattern corresponding to one or more operating states of one or more components thereof or associated therewith. The particle beam emitter 272 can be operatively coupled to one or more proton beam delivery systems to provide one or more proton beams to the proton beam delivery systems. The particle beam emitter 272 can operate in accordance with at least one clinical therapy, proton beam therapy, radiation therapy, or the like. The particle beam emitter 272 is not limited to emitting protons, as discussed herein by way of example. The interface controller 274 can correspond at least partially in one or more of structure and operation to the interface controller 212. For example, the interface controller 274 can link the particle emitter system 203 with one or more of the network 201 and the data processing system 204 by one or more communication interfaces.

The client system 204 can include a computing system associated with a database system. For example, the client system 204 can correspond to a cloud system, a server, a distributed remote system, or any combination thereof. For example, the client system 204 can include an operating system to execute a virtual environment. The operating system can include hardware control instructions and program execution instructions. The operating system can include a high-level operating system, a server operating system, an embedded operating system, or a bootloader. The client system 204 can include a user interface device 280 and an interface controller 282.

The user interface device 280 can include one or more devices to receive input from a user or to provide output to a user. For example, user interface 280 can correspond to a display device that provides visual output to a user and one or more user input devices that receive input from a user. For example, the input devices can include a keyboard, mouse, or touch-sensitive panel of the display device but are not limited thereto. The display device can display at least one or more presentations, as discussed herein, and can include an electronic display. An electronic display can include, for example, a liquid crystal display (LCD), a light-emitting diode (LED) display, an organic light-emitting diode (OLED) display, or the like. The display device can receive, for example, capacitive or resistive touch input. The display device can be housed at least partially within the client system 204.

The Interface Controller 282 can correspond at least partially in one or more of the structures and operations to the Interface controller 212. For example, the interface controller 282 can link the client system 204 with one or more of the network 201 and the data processing system 204, by one or more communication interfaces.

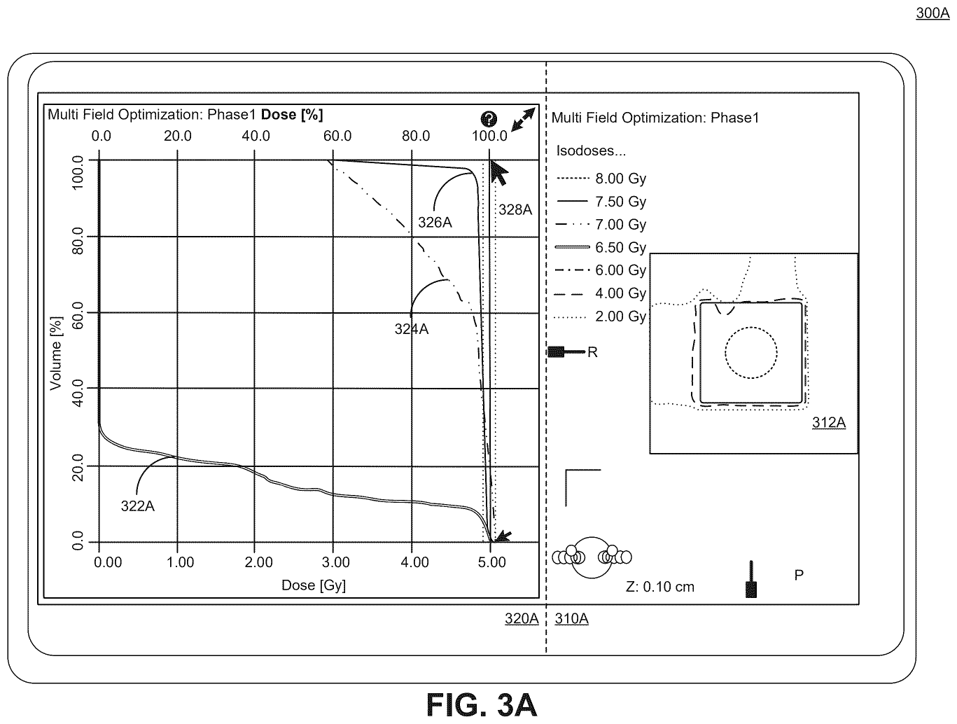

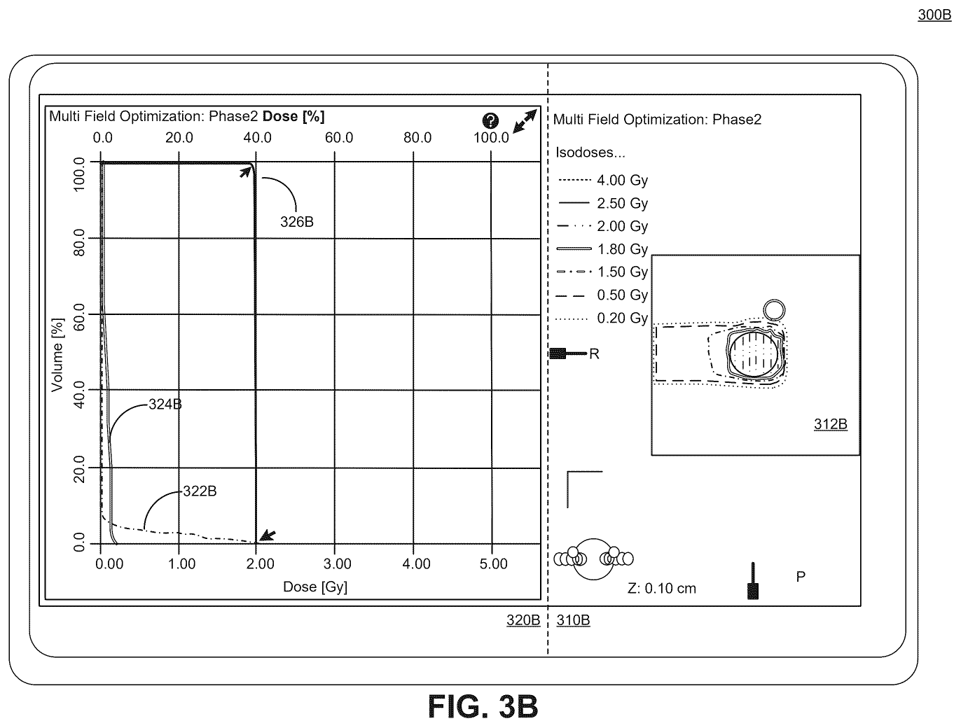

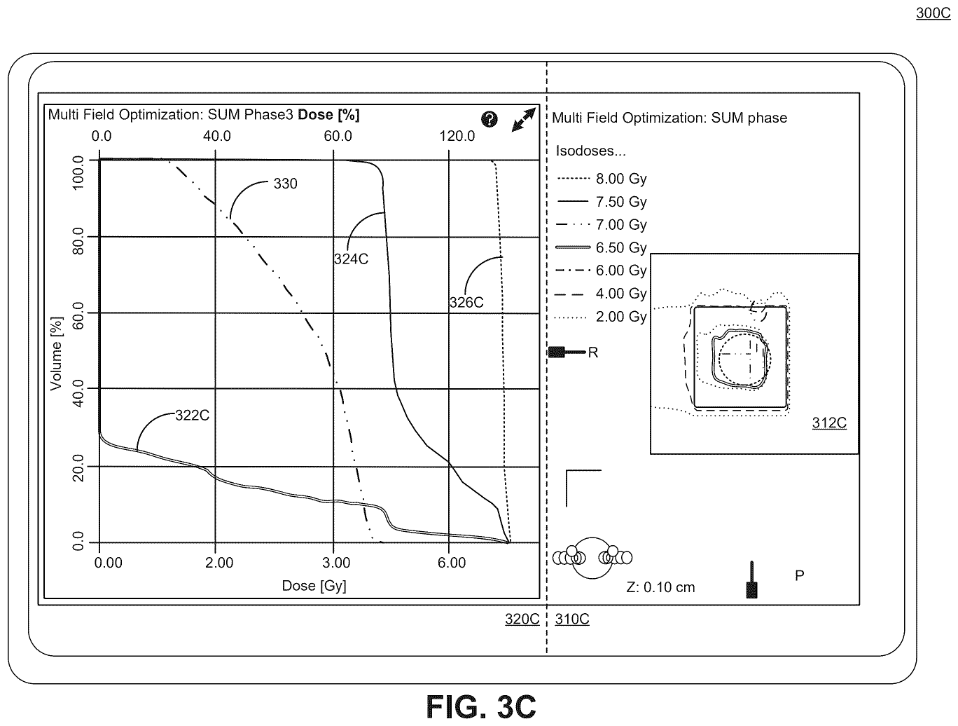

FIGS. 3A-C illustrate a non-limiting example of the multi-phase optimization control-feedback system. A user may switch between the depicted three views. Each view may contain the objectives and doses for that particular phase. As depicted, Phase 1 and Phase 2 (FIG. 3A-B) match the treatment plans, and the “SUM” view (FIG. 3C) depicts a virtual plan that is calculated from the two other plans transiently. The objectives set for the SUM plan may be applied against the dose sums.

In a non-limiting example, each phase discussed herein may be “self-contained. ” That is, in some embodiments, a processor can optimize each phase independently and/or their sum, as if each phase (or the summation results) is a normal “single-phase” treatment plan. As such, each phase can, for example, contain objectives for the dose distribution of individual anatomical structures and, in case of proton plans, for the proton spot weights. In some embodiments, such as the one discussed herein, the first phase may contain upper and lower dose objectives for the Elective Volume (e.g., 326A and the DVH objective 328A). Likewise, the second phase may contain objectives for the Boost Volume (e.g., 326B). Finally, the Phase Sum may contain one upper mean dose objective for the OAR. The mean dose objective can be represented as a diamond shape on the X-axis. In the depicted embodiment, the appropriate location may be around 2.75 Gy. With the aforementioned objectives, both Phase 1 and 2 may fulfill the required Elective/Boost target coverage, while the total (sum) dose to the OAR is being limited.

The methods and systems discussed herein may use a multi-part paradigm to generate treatment attributes. For instance, one or more processors can have a control phase and a feedback phase. The control phase (sometimes referred to as the first phase) may involve a user interface that allows users to define optimization objectives for each phase of treatment and for the combined plan sum. This interface may provide separate views for each phase (Phase 1 and Phase 2) and the sum of the phases (SUM). In some embodiments, each view enables the user to set specific dose objectives and control parameters for that particular phase or the overall treatment. The interface may allow for seamless switching between different phases and the combined view, facilitating the setting and adjustment of objectives without needing to exit the optimization process.

The second phase (sometimes referred to herein as the feedback phase) may provide real-time information on how well the system meets the defined optimization objectives. This phase may include visual feedback such as dose-volume histograms (DVHs) and dose distributions for each phase and the combined plan. Each view may illustrate the feedback for the respective phase or the sum of the phases, allowing users to see the current dose statistics and how the optimization process is progressing. This feedback loop may help users assess the impact of their adjustments to the control parameters and make informed decisions to achieve the desired dose distribution.

Together, the control and feedback phases may create a self-contained optimization dialogue, enabling efficient and effective treatment planning. Users can define what they want from each phase and the overall treatment while simultaneously receiving feedback on how well those objectives are being met. This integration minimizes the need for iterative re-planning and ensures that both individual phase objectives and combined dose objectives are optimally balanced.

The proposed system addresses the inefficiencies of traditional multi-phase treatment planning by enabling simultaneous optimization of both phases using both phase-specific and sum objectives, as illustrated in FIGS. 3A-C. Each phase may be assigned its own objectives for the target structures, while the objectives for the organs at risk (OAR) are assigned to the combined plan sum rather than to individual phases. During the optimization process, the user interface allows for toggling between views of the different phases and the sum plan. This enables users to set and evaluate objectives for each phase and the overall plan without exiting the optimization dialogue.

The optimization algorithm may process all treatment fields and the associated phase information. It receives the objectives and determines which phase or combined plan they pertain to. Patient data, such as the structure set, may be consistent across phases and only needs to be input once. The processors may calculate the dose for each treatment field and use these calculations to construct the dose for each phase. The phase doses may then be combined to form the sum dose. The algorithm used by the processors may evaluate the objectives against the corresponding phase or sum dose and minimize the global cost by adjusting the treatment fields accordingly. During the optimization, the algorithm may provide objective costs and dose matrices for each phase and the combined plan, updating all views in the user interface in real-time. In the optimization dialogue, depicted in FIGS. 3A-C, users can view DVHs, objectives, and current dose statistics for each specific phase (Phase 1 and Phase 2), as well as the total dose from both phases (SUM). For instance, the total dose delivered to PTV2 can be easily seen to be 7 Gy with good homogeneity while ensuring that the OAR does not receive an excessive dose.

The multi-phase approach discussed herein may streamline the treatment planning process by integrating the optimization of multiple phases into a single workflow. This reduces the need for iterative re-planning and improves the overall efficiency and effectiveness of radiation therapy planning.

In some embodiments, different phases can be optimized based on a particular treatment attribute. Therefore, each dose will include its own objectives, while the OAR objective may be assigned to the plan sum instead of either of the phases.

FIG. 3A depicts an example user interface presentation of first phase optimization, according to this disclosure. As illustrated by way of example in FIG. 3A, a user interface presentation of first phase optimization 300A can include at least a target frame presentation 310A, and a phase optimization presentation 320A. For instance, a DVH of different organs/structures can be presented as depicted. The DVH may include dose distribution information to different organs in a graph form, such that the user can visually determine whether the dose distributions are within defined tolerances. For instance, line 322A may indicate the dose received by an OAR, while lines 234A and 236A represent dose distributions to other structures (another OAR and the PTV, respectively). The target frame presentation 310A can provide information regarding the plan optimization. For instance, the frame presentation may provide data that can be viewed by the end-user regarding various objectives and treatment attributes. The target frame presentation 310A can include dose delivery presentations 312A, which may provide a visual depiction of the PTV and/or OARs.

With respect to FIG. 3A, the line 326A (e.g., Phase 1 target structure, which is sometimes referred to as “Elective volume”) corresponds to the structure 130A in FIG. 1A, line 324A (e.g., OAR, for whom the dose from Phase Sum is being optimized/minimized) corresponds to the structure 112 in FIG. 1A. Moreover, the line 322A (e.g., body structure) may correspond to the structure 120A in FIG. 1A. The dose to this structure may not be optimized in the example, but the DVH may provide useful information to the user utilizing the methods and systems discussed herein to optimize a treatment plan.

FIG. 3B depicts an example user interface presentation of second-phase optimization, according to this disclosure. As illustrated by way of example in FIG. 3B, a user interface presentation of second phase optimization 300B can include at least a target frame presentation 310B and a phase optimization presentation 320B. The second phase optimization 300B is similar to the first phase 300A. However, in the second phase optimization 300B, the processor is instructed to optimize different treatment attributes.

With respect to FIG. 3B, the lines represent DVHs based on the Phase 2 of dose optimization. In this figure, line 326B may correspond to Phase 2 target structure, referred to as “Boost volume” herein. The line 3626B may correspond to the structure 130B in FIG. 1B. Lines 324B and 322B correspond to lines 324A and 322A respectively (same structures as FIG. 1A).

In the second phase optimization 300B, the processor uses an optimization algorithm that may predict different dosages applied to different organs/structures, as depicted in the DVH presented in the phase optimization presentation 320B. Specifically, lines 322B, 324B, and 326B provide significantly different dosage predictions for the organs/structures of the patient. In some embodiments, the same organs as FIG. 3A are depicted. This is because the second phase optimization uses different user-defined attributes. As a result, the results predicted by the optimization models are different. Similar to the target frame presentation 310A, the target frame presentation 310B provides detailed data indicating information predicted by the processors (optimization models). Moreover, the dose delivery presentation 312B provides a visual depiction of the PTV and/or OARs.

FIG. 3C depicts an example user interface presentation of summation-of-phases optimization, according to this disclosure. As illustrated by way of example in FIG. 3C, a user interface presentation of second phase optimization 300C can include at least a target frame presentation 310C, and a phase optimization presentation 320C. The phase optimization presentation 320C may provide a view that is a combination of the first phase (FIG. 3A) and the second phase (FIG. 3B). Therefore, the DVH presented in the phase optimization presentation 320C provides a combination of doses as calculated in the first and second phases (including lines 322C, 324C, and 326C). Similarly, the dose delivery presentations 312C is a combination of dose delivery presentations 312A and dose delivery presentations 312B. Moreover, the target frame presentation 310C may present overall results such that the combined phases are compared against the overall clinical goals and objectives.

In FIG. 3C, the line 326C may correspond to Phase 2 target structure, referred to sometimes as the “Boost Volume;” line 324C may correspond to Phase 1 target structure, sometimes referred to as the “Elective Volume;” and the line 330 may correspond to the OAR; and finally, the line 322C may correspond to the body.

In some embodiments, different phases can be optimized based on a particular treatment attribute. Therefore, each dose will include its own objectives, while the OAR objective may be assigned to the plan sum instead of either of the phases.

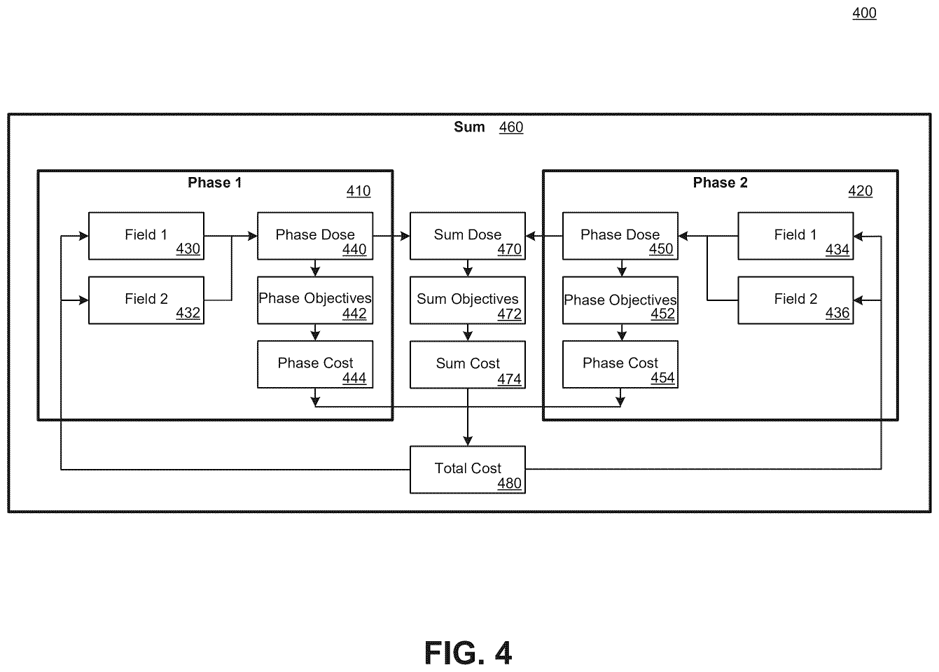

FIG. 4 depicts an example computer architecture for dose optimization, according to this disclosure. FIG. 4 illustrates the data flow within the multi-phase optimization algorithm, showing how it processes treatment fields and optimization objectives for each phase, calculates the corresponding doses, and combines these to form a sum dose. The algorithm evaluates how well the calculated doses meet the objectives for both individual phases and the combined plan, adjusting the treatment fields iteratively to minimize the global cost and ensure optimal dose distribution across the phases.

As illustrated by the example in FIG. 4, a computer architecture for dose optimization 400 can include at least a first-phase optimization architecture 410, a second-phase optimization architecture 420, and a combined radiation summation architecture 460. Each phase optimization architecture can be executed separately at the same time (in parallel) or consecutively. Objectives and fields for both phases, as well as objectives for the sum of phases, are given as inputs (e.g., by the user). The dose for each phase is calculated from the fields of that phase, and the sum dose is subsequently calculated from the phase doses. The objectives for each phase/sum are evaluated against the dose of the phase/sum, and the fields are adjusted to yield doses that would minimize the total cost (phase costs plus sum cost).

The dose optimization 400 illustrates how the optimization algorithm operates. The dose optimization 400 starts when a user identifies the fields 430 and 432 (for Phase 1) and files 434 and 436 (for Phase 2). The user may then provide plan objectives (442 for Phase 1 and 452 for Phase 2). The dose optimization 400 then uses various internal rule datasets or plan optimizers to generate phase dose 440 for Phase 1 and phase dose 450 for Phase 2. As used herein, the phase dose is a prediction of dosage implemented for different structures using the parameters and attributes (inputted by the user).

The dose optimization 400 may then evaluate each phase dose and generate a phase cost 444 and 454. As used herein, the phase cost (444 or 454) refers to a value calculated by the optimization algorithm that quantifies how well the dose distribution for a specific phase meets its defined optimization objectives. During the optimization process, each phase has specific targets for the radiation dose to be delivered to the target structures and objectives (sometimes referred to as constraints as well) to limit the dose to OAR. Accordingly, the phase cost may represent the degree of discrepancy between the actual dose distribution and these objectives. The optimization algorithm may aim to minimize this phase cost by adjusting the treatment fields within the phase. A lower phase cost indicates that the dose distribution closely aligns with the set objectives, while a higher phase cost signifies a greater deviation. By iterating adjustments to minimize the phase cost, the algorithm ensures that the treatment plan for each phase meets its specific goals as effectively as possible.

After each phase is evaluated independently, the processors may combine the predicted doses. Specifically, during the combined radiation summation architecture 460, the processors may combine the treatment attributes identified in each phase through a series of steps designed to ensure that both individual phase objectives and combined objectives are met optimally. Initially, the processors may receive inputs, including the treatment fields and optimization objectives for each phase, encompassing the dose distribution goals for the target structures and objective for the OAR for each phase.

For each phase, the processor may calculate the summed dose distribution 470 based on the treatment fields, determining the radiation dose delivered to the target volumes and OARs. After calculating the dose for each phase, the processor may construct the summed dose 470 by combining the dose distributions from all phases and aggregating the individual phase doses to get a total dose distribution that encompasses all phases of the treatment.

The processor then evaluates how well the calculated phase doses and the summed dose 470 meet the defined optimization objectives (e.g., summed objectives 472). This includes checking the compliance of each phase with its specific goals and the combined plan with overall objectives, particularly for the OARs. Following this, the processor may calculate the cost for each phase and the sum cost, representing the discrepancy between the actual dose distribution and the objectives. The phase cost reflects the specific phase's goals, while the sum cost accounts for the overall treatment objectives. The summation of the costs may be represented as summed cost 474.

Using the calculated summed costs 474, the processor may iteratively adjust the treatment fields to minimize these costs, revising the radiation parameters to better align the dose distributions with the optimization objectives for both the individual phases and the combined plan. Throughout this process, the processor may update the user interface views with the latest objective costs and dose matrices for each phase and the summation of the phases. This real-time or near-real-time feedback allows users to monitor progress and make informed adjustments as needed. Therefore, after the processor optimizes the treatment plan, the processor may generate a total cost 480 for the treatment plan, which represents the cost of treating the patient via the attributes predicted in each phase. The total cost 480 may be the summation of all (both phases and Sum phase) costs. The total cost 480 is the cost that the optimizer seeks to minimize by adjusting the fields.

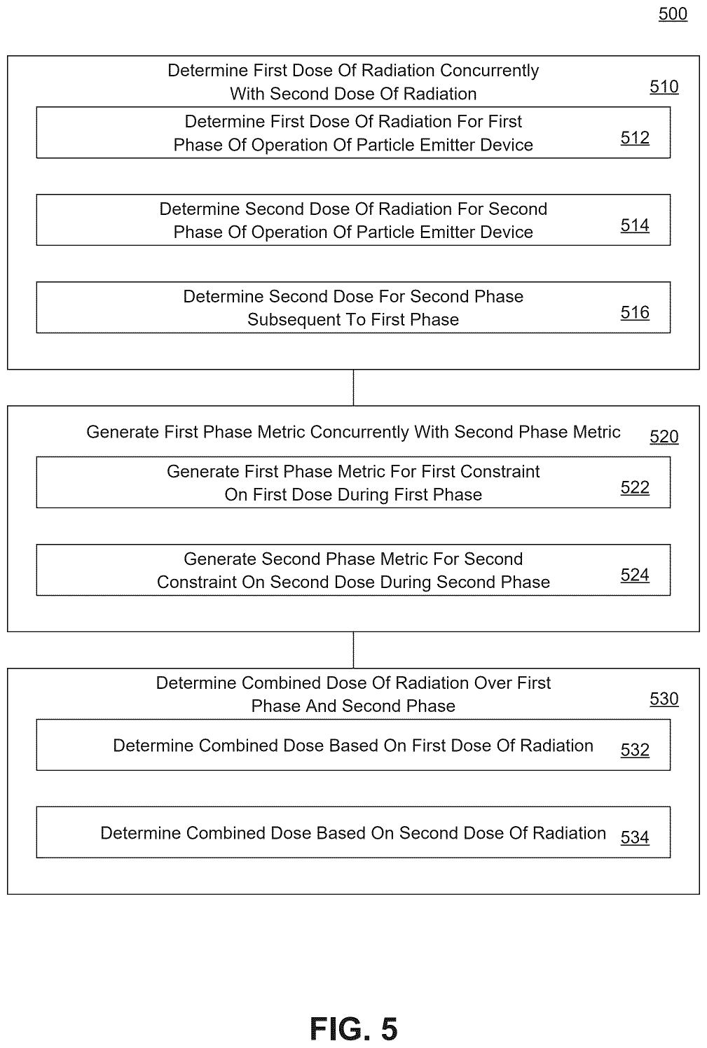

FIG. 5 depicts an example method of concurrent optimization of particle emitter systems for multi-phased treatment, according to this disclosure. At least one of the user interfaces of FIGS. 1A-B, 3A-C, the system of FIG. 2, the architecture of FIG. 4, or any component thereof, or any combination thereof, can perform method 500. At 510, the method 500 can determine a first dose of radiation concurrently with a second dose of radiation. At 512, the method 500 can determine the first dose of radiation for a first phase of operation of a particle emitter device. At 514, the method 500 can determine the second dose of radiation for a second phase of operation of a particle emitter device. At 516, the method 500 can determine the second dose for a second phase subsequent to the first phase. At 520, the method 500 can generate a first phase metric concurrently with a second phase metric. At 522, the method 500 can generate the first phase metric for a first objective on the first dose during the first phase. At 524, the method 500 can generate the second phase metric for a second objective on the second dose during the second phase. At 530, the method 500 can determine a combined dose of radiation over the first phase and the second phase. At 532, the method 500 can determine the combined dose based on the first dose of radiation. At 534, the method 500 can determine the combined dose based on the second dose of radiation.

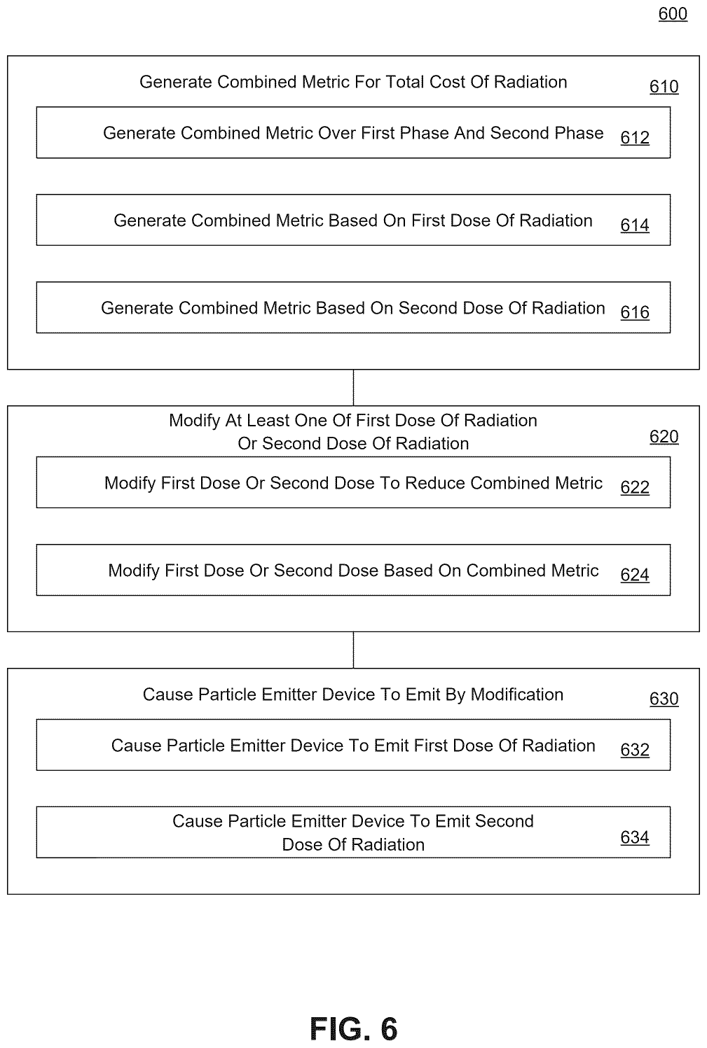

FIG. 6 depicts an example method of concurrent optimization of particle emitter system for multi-phased treatment, according to this disclosure. At least one of the user interfaces of FIGS. 1A-B, 3A-C, the system of FIG. 2, the architecture of FIG. 4, or any component thereof, or any combination thereof, can perform method 600.

At 610, the method 600 can generate a combined metric for a total cost of radiation. In an aspect, the method can include generating, based on the second combined metric, the combined metric corresponding to the total cost of radiation. In an aspect, the system can generate, based on the second combined metric, the combined metric corresponding to the total cost of radiation. At 612, the method 600 can generate the combined metric over the first phase and the second phase. At 614, the method 600 can generate the combined metric based on the first dose of radiation. At 616, the method 600 can generate the combined metric based on the second dose of radiation. In an aspect, the method can include generating, based on the combined dose of radiation and a third objective on the combined dose of radiation, a second combined metric corresponding to a cost of radiation independent of the first phase and the second phase. In an aspect, the system can generate, based on the combined dose of radiation and a third objective on the combined dose of radiation, a second combined metric corresponding to a cost of radiation independent of the first phase and the second phase.

At 620, the method 600 can modify at least one of the first dose of radiation or the second dose of radiation. In an aspect, the method can include modifying at least one of the first dose of radiation or the second dose of radiation to reduce the combined metric over one or more subsequent iterations. In an aspect, the system can modify at least one of the first dose of radiation or the second dose of radiation to reduce the combined metric over one or more subsequent iterations. At 622, the method 600 can modify the first dose or the second dose to reduce the combined metric. At 624, the method 600 can modify the first dose or the second dose based on the combined metric.

At 630, the method 600 can cause the particle emitter device to emit according to the modification. At 632, the method 600 can cause the particle emitter device to emit the first dose of radiation. In an aspect, the particle emitter device is configured to provide, during the first phase, the first dose of radiation via a first plurality of fields. At 634, the method 600 can cause the particle emitter device to emit the second dose of radiation. In an aspect, the particle emitter device is configured to provide, during the second phase, the second dose of radiation via a second plurality of fields.

In an aspect, the method can include causing a user interface to present one or more first indications of the first dose of radiation over the one or more subsequent iterations. The method can include causing the user interface to present one or more second indications of the second dose of radiation over the one or more subsequent iterations overlaid with the one or more first indications. In an aspect, the system can cause a user interface to present one or more first indications of the first dose of radiation over the one or more subsequent iterations. The system can cause the user interface to present one or more second indications of the second dose of radiation over the one or more subsequent iterations overlaid with the one or more first indications. In an aspect, the method can include causing the user interface to present one or more third indications of the combined dose of radiation over the one or more subsequent iterations overlaid with the one or more first indications and the one or more second indications. In an aspect, the system can cause the user interface to present one or more third indications of the combined dose of radiation over the one or more subsequent iterations overlaid with the one or more first indications and the one or more second indications.

Having now described some illustrative implementations, the foregoing is illustrative and not limiting, having been presented by way of example. In particular, although many of the examples presented herein involve specific combinations of method acts or system elements, those acts, and those elements may be combined in other ways to accomplish the same objectives. Acts, elements, and features discussed in connection with one implementation are not intended to be excluded from a similar role in other implementations.

The phraseology and terminology used herein are for the purpose of description and should not be regarded as limiting. The use of “including,” “comprising,” “having,” “containing,” “involving,” “characterized by,” “characterized in that,” and variations thereof herein is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items. References to “is” or “are” may be construed as nonlimiting to the implementation or action referenced in connection with that term. The terms “is” or “are” or any tense or derivative thereof, are interchangeable and synonymous with “can be” as used herein, unless stated otherwise herein.

Directional indicators depicted herein are example directions to facilitate understanding of the examples discussed herein, and are not limited to the directional indicators depicted herein. Any directional indicator depicted herein can be modified to the reverse direction, or can be modified to include both the depicted direction and a direction reverse to the depicted direction, unless stated otherwise herein. While operations are depicted in the drawings in a particular order, such operations are not required to be performed in the particular order shown or in sequential order, and all illustrated operations are not required to be performed. Actions described herein can be performed in a different order. Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any clam elements.

Scope of the systems and methods described herein is thus indicated by the appended claims, rather than the foregoing description. The scope of the claims includes equivalents to the meaning and scope of the appended claims.

What is claimed is:

1. A system, comprising:
one or more processors, coupled with memory, to:
determine a first dose of radiation concurrently with a second dose of radiation, the first dose of radiation associated with a first phase of operation of a particle emitter device, and the second dose of radiation associated with a second phase of operation of a particle emitter device, the second phase subsequent to the first phase;
generate a first phase metric concurrently with a second phase metric, the first phase metric corresponding to a first objective on the first dose of radiation during the first phase, and the second phase metric corresponding to a second objective on the second dose of radiation during the second phase;
determine, based on the first dose of radiation and the second dose of radiation, a combined dose of radiation over the first phase and the second phase;
generate, based on the first dose of radiation and the second dose of radiation, a combined metric corresponding to a total cost of radiation over the first phase and the second phase;
modify, based on the combined metric, at least one of the first dose of radiation or the second dose of radiation to reduce the combined metric; and
cause the particle emitter device to emit the first dose of radiation and the second dose of radiation according to the modification.

2. The system of claim 1, the processors to:
generate, based on the combined dose of radiation and a third objective on the combined dose of radiation, a second combined metric corresponding to a cost of radiation independent of the first phase and the second phase.

3. The system of claim 2, the processors to:
generate, based on the second combined metric, the combined metric corresponding to the total cost of radiation.

4. The system of claim 1, the processors to:
modify at least one of the first dose of radiation or the second dose of radiation to reduce the combined metric over one or more subsequent iterations.

5. The system of claim 1, the processors to:
cause a user interface to present one or more first indications of the first dose of radiation over the one or more subsequent iterations; and
cause the user interface to present one or more second indications of the second dose of radiation over the one or more subsequent iterations overlaid with the one or more first indications.

6. The system of claim 5, the processors to:
cause the user interface to present one or more third indications of the combined dose of radiation over the one or more subsequent iterations overlaid with the one or more first indications and the one or more second indications.

7. The system of claim 1, wherein the particle emitter device is configured to provide, during the first phase, the first dose of radiation via a first plurality of fields.

8. The system of claim 7, wherein the particle emitter device is configured to provide, during the second phase, the second dose of radiation via a second plurality of fields.

9. A method, comprising:
determining a first dose of radiation concurrently with a second dose of radiation, the first dose of radiation associated with a first phase of operation of a particle emitter device, and the second dose of radiation associated with a second phase of operation of a particle emitter device, the second phase subsequent to the first phase;
generating a first phase metric concurrently with a second phase metric, the first phase metric corresponding to a first objective on the first dose of radiation during the first phase, and the second phase metric corresponding to a second objective on the second dose of radiation during the second phase;
determining, based on the first dose of radiation and the second dose of radiation, a combined dose of radiation over the first phase and the second phase;
generating, based on the first dose of radiation and the second dose of radiation, a combined metric corresponding to a total cost of radiation over the first phase and the second phase;
modifying, based on the combined metric, at least one of the first dose of radiation or the second dose of radiation to reduce the combined metric; and
causing the particle emitter device to emit the first dose of radiation and the second dose of radiation according to the modification.

10. The method of claim 9, further comprising:
generating, based on the combined dose of radiation and a third objective on the combined dose of radiation, a second combined metric corresponding to a cost of radiation independent of the first phase and the second phase.

11. The method of claim 10, further comprising:
generating, based on the second combined metric, the combined metric corresponding to the total cost of radiation.

12. The method of claim 9, further comprising:
modifying at least one of the first dose of radiation or the second dose of radiation to reduce the combined metric over one or more subsequent iterations.

13. The method of claim 9, further comprising:
causing a user interface to present one or more first indications of the first dose of radiation over the one or more subsequent iterations; and
causing the user interface to present one or more second indications of the second dose of radiation over the one or more subsequent iterations overlaid with the one or more first indications.

14. The method of claim 13, further comprising:
causing the user interface to present one or more third indications of the combined dose of radiation over the one or more subsequent iterations overlaid with the one or more first indications and the one or more second indications.

15. The method of claim 9, wherein the particle emitter device is configured to provide, during the first phase, the first dose of radiation via a first plurality of fields.

16. The method of claim 15, wherein the particle emitter device is configured to provide, during the second phase, the second dose of radiation via a second plurality of fields.

17. A non-transitory computer readable medium including one or more instructions stored thereon and executable by a processor to:
determine, by the processor, a first dose of radiation concurrently with a second dose of radiation, the first dose of radiation associated with a first phase of operation of a particle emitter device, and the second dose of radiation associated with a second phase of operation of a particle emitter device, the second phase subsequent to the first phase;
generate, by the processor, a first phase metric concurrently with a second phase metric, the first phase metric corresponding to a first objective on the first dose of radiation during the first phase, and the second phase metric corresponding to a second objective on the second dose of radiation during the second phase;
determine, by the processor and based on the first dose of radiation and the second dose of radiation, a combined dose of radiation over the first phase and the second phase;
generate, by the processor and based on the first dose of radiation and the second dose of radiation, a combined metric corresponding to a total cost of radiation over the first phase and the second phase;
modify, by the processor and based on the combined metric, at least one of the first dose of radiation or the second dose of radiation to reduce the combined metric; and
cause, by the processor, the particle emitter device to emit the first dose of radiation and the second dose of radiation according to the modification.

18. The non-transitory computer readable medium of claim 17, further including one or more instructions executable by the processor to:
generate, by the processor and based on the combined dose of radiation and a third objective on the combined dose of radiation, a second combined metric corresponding to a cost of radiation independent of the first phase and the second phase.

19. The non-transitory computer readable medium of claim 17, further including one or more instructions executable by the processor to:
generate, by the processor and based on the second combined metric, the combined metric corresponding to the total cost of radiation.

20. The non-transitory computer readable medium of claim 17, further including one or more instructions executable by the processor to:
modify, by the processor, at least one of the first dose of radiation or the second dose of radiation to reduce the combined metric over one or more subsequent iterations.
